(19) United States
(12) Patent Application Publication
(10) Pub. No.: US 2008/0009952 A1
(43) Pub. Date: Jan. 10, 2008

(54) Precision acetabular machining system and resurfacing acetabular implant

(76) Inventor: W. Andrew Hodge, Boynton Beach, FL (US)

Correspondence Address: MCHALE & SLAVIN, P.A., 2855 PGA BLVD, PALM BEACH GARDENS, FL, 33410, US

(21) Appl. No.: 11/772,698

(22) Filed: Jul. 2, 2007

Related U.S. Application Data
(60) Provisional application No. 60/806,376, filed on Jun. 30, 2006.
(60) Provisional application No. 60/806,399, filed on Jun. 30, 2006.

(51) Int. Cl. A61F 2/34

(52) U.S. Cl. 623/22.21

(57) ABSTRACT

This application is directed toward a quantitative high precision acetabular machining system designed to enable accurate and precise positioning of acetabular resurfacing implants, or the like bone socket resurfacing devices, in a manner which eliminates malpositioning, loss of center, drift and inappropriate depth calculations. Subsequent to site preparation, a precision implant is disclosed which provides for resurfacing of damaged articular cartilage with bone preserving and non-drift instrumentation utilizing synthetic materials in an anatomically correct fashion to maintain excellent hip stability while maintaining an accurate hip joint center mated with anatomic femoral head sizes.

### CROSS-REFERENCE TO RELATED APPLICATIONS

This invention relies upon the filing dates of U.S. Provisional Application 60/806,376, file Jun. 30, 2006, and U.S. Provisional Application 60/806,399, file Jun. 30, 2006, the contents of which are each herein incorporated by reference in their entirety.

### FIELD OF THE INVENTION

This invention relates to a precision acetabular machining system; particularly to a system for preparing a natural acetabulum for a resurfacing implant; and most particularly to a combination of elements which concurrently utilize a centralized guide hole concept to enable precise preparation, implant positioning, and component fixation.

### BACKGROUND OF THE INVENTION

As a result of, for example, disease or trauma, a patient may require a hip replacement procedure, which may involve a total hip replacement or a partial hip replacement. In a total hip replacement procedure, a femoral component having a head portion is utilized to replace the natural head portion of the thighbone or femur. The femoral component typically has an elongated intramedullary stem which is utilized to secure the femoral component to the patient's femur. In such a total hip replacement procedure, the natural bearing surface of the acetabulum is resurfaced or otherwise replaced with a cup-shaped acetabular component that provides a bearing surface for the head portion of the femoral component.

There are a variety of known prior art techniques for securing the acetabular cups. For example, acetabular cups may be secured to the acetabulum by the use of bone cement. Alternatively, it may be desirable to secure artificial components to natural bone structures without the use of bone cement. For this reason, the prior art has suggested a number of press fit acetabular cups designed for securement to the acetabulum without the use of bone cement. Regardless of the favored system, it is nevertheless required that the acetabulum is first reamed by the surgeon in order to create a cavity into which the acetabular cup is secured by the use of a surgical tool known as a reamer. It is often difficult for the surgeon to properly match the size of the reamer to the desired acetabular cup size. In point of fact, due to the variations in bone hardness within the acetabulum that is to be reamed, the presently available reaming devices often result in a somewhat oblong hole.

Prior art efforts to enable good socket fixation in such poorly prepared acetabulum have led to a plethora of compensating designs, for example a number of acetabular cups have been designed with a flared rim (known as dual radius or “bubble” cups) or a frusto-conically shaped annular rim portion (known as dual-geometry cups). Although the configuration of such cups may generate relatively strong retention forces at the rim portion of the cup, surface contact and therefore retention forces are relatively small at the portions of the outer shell other than the rim portion, particularly in the dome area. Moreover, such reduced surface contact at the portions of the outer shell other than the rim portion reduces bone ingrowth in such portions.

This invention relates to improvements in the performance and outcome of hip arthroplasty, particularly to a quantitative high precision acetabular machining system (PAMS) designed to enable accurate and precise positioning of acetabular resurfacing implants, or the like bone socket resurfacing devices, in a manner which eliminates malpositioning, loss of center, drift and inappropriate depth calculations, and furthermore to a precision fit resurfacing acetabular implant which, in conjunction with use of the precision acetabular machining system, enables performance of a bone sparing procedure resulting in a stable joint and lowered incidence of acetabular protrusion.

Prior art practice has called for the use of standard hip replacement sockets, whose use concomitantly requires the unwanted sacrificing of significant bone, resulting in undesirable inaccuracies in positioning and joint center drift. The lack of accurate and precise machining techniques for preparation of the acetabulum requires additional engineering of the socket components to better enable successful positioning and adequate fixation. Such compensation includes the use of peripheral lips; oversized, thicker walled cups and non-hemisperical shaped inserts; and furthermore requires the surgeon to specify smaller femoral head sizes. In order to avoid the high rate of acetabular protrusion (a 50% rate of acetabular protrusion after 4 years), cup spin-out (loosening) and malpositioning, a new implant, instrumentation, and operative technique are required which will provide for anatomic hip resurfacing to reinforce the socket side with bipolar or unipolar procedures.

Likewise, current hip socket reaming instruments are unguided, hemispherical bone cutting devices which are not calibrated for depth. They are used on a standard, hand held power drill/reamer, and are subject to unacceptable reaming inaccuracies which in turn lead to implant malpositions, loosening, high articular wear rates, and hip dislocations.

The conventional THR socket requires substantial deepening of the natural arthritic socket by approximately 5-10 mm, destroying valuable bone and decreasing hip leverage (offset) by loosing the abductor lever arm. Use of existing reaming systems is problematic, given that the reaming often results in formation of an oblong, out of round hole, due to the posterior bone being quite hard while the anterior bone is much softer causing the presently available reamers to drift anteriorly.

Furthermore, surgeons have traditionally used a two-dimensional X-ray to estimate the three dimensional acetabular depth leading to frequent malpositioning and common over reaming with excessive bone loss. Third, with many monoblock large head sockets coming on the market, the surgeon does not have any hole(s) in the implant to judge proper seating, so there is greater need for precise reaming. Fourth, with oblong bone preparation the surgeon has lack of implant fixation which drifts as attempts of further seating are performed and sometimes leads to bone fracture as well as implant malpositioning which can cause edge loading, high wear rates and dislocations of the hip.

The standard sockets also have used traditional biomaterials usually requiring a 28 mm femoral head or smaller. This can lead to instability problems of the hip and also result in an increased amount of wear particles with conventional materials.

Additionally, with hip hemiarthroplasty, especially in patients under 75 years old, there is a high incidence of acetabular protrusion and pain due to aggressive cartilage wear by metal heads.

The PAMS instrumentation solves all of the above currently existing problems of socket reaming systems. PAMS establishes the anatomic socket center, then drills a centering hole which precisely guides new reamers in a concentric, quantitative fashion. This gives the surgeon the knowledge and security to accurately machine the socket bone with maximal conservation and optimal acetabular implant seating and positioning.

In light of the precision reaming, unnecessary bone loss is avoided, paving the way for providing a precisely sized resurfacing implant, which eliminates acetabular protrusion and chronic pain, and enables a much thinner implant having a high degree of fixation, and permitting use of larger femoral head sizes, and realizing a long felt need in the art.

### DESCRIPTION OF THE PRIOR ART

Rehder, U.S. Pat. No. 4,271,849, discloses an apparatus for producing relief grooves in pan-shaped bones, especially in the acetabulum of a human hip joint. The apparatus includes a drivable drive shaft having a free end section. A casing is arranged on the free end section. The casing has an exterior contour which, facing away from the drive shaft is, at most, as large as the pan-contour of the bone. At least one cutting device is arranged in the casing which is movable transversely relative to the longitudinal axis of the drive shaft. The cutting device has a cutting edge arranged in an outwardly radial direction. An adjusting device is included which cooperates with the cutting device. The cutting edge is arranged in an initial position within the casing and is radially movable outwardly by means of the adjusting device.

Matsen, III, et al., U.S. Pat. No. 5,030,219, relates to instruments for preparing a glenoid surface of a scapula to receive a prosthetic glenoid component to replace the natural socket of a shoulder. More particularly, the invention of Masten, III et al relates to a drill guide assembly for aligning and guiding a drill bit to form holes in the glenoid surface at predetermined locations to secure the glenoid component to the glenoid surface. A reamer assembly including a ratchet drive mechanism is also included to facilitate preparation of the glenoid surface prior to installation of the glenoid component.

Allard et al., U.S. Pat. No. 6,245,074, discloses an orthopaedic reamer including an elongate shaft and a cutting head attached to an end of the shaft. The cutting head has a diameter which is larger than the shaft. The cutting head has a radial perimeter and an axial cutting face with a plurality of cutting teeth. The cutting head has at least one visualization groove which extends radially inward from the radial perimeter. The at least one visualization groove allows a surgeon to visualize the cut bone during surgery.

Lechot, U.S. Pat. No. 6,702,819, discloses a reamer intended for surgery, which includes a hollow body of revolution provided with four radial arms which are perpendicular to each other so as to form a cross for fixing the reamer on a reamer holder. The cross formed by the radial arms is made up of a first diametral bar (1), a pin (2) passing through the first bar at its center and protruding through each side of this bar, and two radial bars (3, 4) which have an axial hole via which each of these radial bars is engaged on the pin. The construction of Lechot is alleged to be simple to produce, while making it possible to omit welds and to eliminate the cleaning problems inherent to these welds.

Lechot et al., U.S. Publication No. 2004/0049199, discloses a low insertion profile surgical reamer for cutting a bone socket comprises a cutting structure. The cutting structure is rotatable about a longitudinal axis. Unlike the present invention, the structure has a static profile area upon insertion of the reamer into the bone socket and a dynamic profile area generated upon rotation, both profile areas lying transverse to the axis. The static profile area is substantially smaller than the dynamic profile area. The reamer includes centrally located holes (18), allowing it to be fixed to a tool holder. With particular reference to FIG. 11, a slotted drill bit may be attached axially to the reamer.

### SUMMARY OF THE INVENTION

The human acetabulum has varied bone hardness from posterior to anterior and has varied thicknesses making surgical reconstruction complex for the operating surgeon. Therefore a news calibrated, precision machining system has been conceived. This concept utilizes a centering hole with peg and calibrating system which allows the surgeon to machine the pelvic hip socket in a calculated (graduated) and directed fashion leading to reaming the correct depth in a precise concentric fashion. The drilling guide and basket reamer are both fashioned to be essentially hemispherical. The guide is used to lock in the acetabular center and also serves as a drill guide for about a 5 mm drill, for example, which can then drill the outer and inner socket cortices. Next, the bone depth measurement is taken, whereby the desired depth is determined and/or set on the centering bullet guide. This centering hole now serves as a graduated bullet tip which snaps thru the centering guide of each reamer.

A depth gauge is provided to enable the surgeon to continually check the depth of drilling and measure the socket thickness. The hemispherical reamers may be provided with a polar graduated peg, which is set for the correct reaming depth. The polar peg keeps the reamer perfectly centered for concentric hemispherical reaming unlike the current oblong, out of round reaming shape obtained by conventional acetabular reaming. Alternatively, a depth gauge which is couplable with the guide may be provided for periodically checking depth. Subsequent to the concentric hemispherical reaming, the surgeon can then easily seat a hemispherical acetabular implant in a perfect press fit fashion, without having to resort to using an oversize implant which can lead to fracture and other assorted positioning and fixation problems. The hemispherical implant of the present invention, which is generally provided with a centering pin for insertion within the guide hole, is then fixated with excellent stability and complete seating.

The instantly disclosed invention provides for resurfacing of damaged articular cartilage with bone preserving and non-drift instrumentation utilizing synthetic materials in an anatomically correct fashion to maintain excellent hip stability while maintaining an accurate hip joint center mated with anatomic femoral head sizes. Ease of implantation with bone preserving, minimally invasive gauged surgical technique insures a superior long term result. Given its potential for longevity, this implant system could also be used in the younger patients, even a hip fracture patient undergoing bipolar or unipolar femoral head replacement, to prevent the commonly seen protrusion failures, since it would be viewed as minimal acetabular resurfacing.

This precision acetabular machining system (PAMS) serves the surgeon's goals of accurately and safely machining the hip socket into pelvic bone to the correct depth and in a concentric acetabular shape compatible with precise implant positioning and seating. This will lead to stable, long term clinical outcomes with optimal articular wear rates and maximal bone preservation.

Embodying minimally invasive instrumentation, only a calculated small amount of modulated surface reaming is required. In one embodiment the implant has a porous titanium backing, a centering pin for coupling with the centering hole and at least one fixation spike, generally positioned superior or posterior/superior. In an alternative embodiment, holes for one or more screws, e.g., one posterior and one anterior, if needed, may be provided. Although not limited thereto, this articular surface can be composed of cobalt chromium, PEEK, cross-linked polyethylene, or the like. Total implant thickness would vary between about 3 mm and about 6 mm depending on articular surface type. It is contemplated that specific instrumentation could be used to quantitate and center the precise minimal machining of the acetabular subchondral bone.

This uniquely thin acetabular implant works as a surface replacement for the damaged and absent articular cartilage found in severe osteoarthritis of the human hip joint. The low profile design obtains excellent stability by matching with large femoral head designs beginning with about 36 mm heads in a socket of 42 mm and increasing in 2 mm increments on both socket and head up to about 64 mm femoral head and about a 70 mm socket. The stability of this implant and accurate restoration of the joint center is enabled by utilization of instrumentation working off a small (1 mm) centering, depth gauging the central acetabular hole, and providing a guide pin for absolute centering and a secondary protrusion, e.g. a spike and/or screw for eliminating axial rotation.

Accordingly, it is a primary objective of the instant invention to provide a PAMS which works off a bicortical centering hole for all reamers, in combination with a resurfacing acetabular implant which does not require undue sacrifice of bone, thereby preserving valuable acetabular bone stock

It is a further objective to prevent surgical migration of the hip joint center and distorted mechanics via utilization of quantitative and centering instrumentation.

It is yet an additional objective of the instant invention to provide excellent hip joint stability because of accurate restoration of joint center.

It is a further objective of the instant invention to teach a PAMS which quantitates the socket bone depth with depth gauged bullet guides to quantitate bone machining.

It is yet another objective of the instant invention to teach a PAMS which accurately selects and controls the anatomic acetabular center during reaming.

It is a still further objective of the instant invention to provide a PAMS which has pre-set reamer stops which prevent over-reaming.

It is yet an additional objective of the instant invention to teach a PAMS which provides precision, concentric, hemispherical machining of socket bone greatly simplifying accurate implant positioning leading to long term excellent outcomes.

It is still an additional objective to provide a hip arthroplasty which allows for the use of anatomic size femoral heads, whereby further increased joint stability is realized.

It is a still further objective to provide a hip arthroplasty which obtains excellent fixation to hard subchondral bone and prevents loosening.

Yet another objective is to provide low wear hip articulation minimizing osteolysis, thereby allowing the device to last through even a young patient's lifetime.

A still further objective of the instant invention is to provide for accurate implantation with precision centering and gauging instruments which utilize key visual landmarks, and a centering pin, which advantageously couples to the centering guide hole, and permits accurate placement of the implant in terms of abduction and anteversion.

Still a further objective of the instant application is provision of an implant which is constructed to provide selectable version by rotation about the centering pin.

A still further objective is to provide an implant wherein additional fixation and limiting of axial rotation are provided by providing internal fixation means integral with the centering pin.

Yet an additional objective of the instant invention is to provide a kit for precision hip arthroplasty comprising, in combination a precision reaming system and cooperative implant system for insuring efficient preparation, placement, and fixation.

Other objects and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a perspective view of a human pelvis showing the acetabular region and particularly the foveal notch for determining the anatomical center for resurfacing;

FIG. 2 shows numerous components of the precision reaming system of the instant invention;

FIG. 3 illustrates the components and relationship used in the initial forming of the centering guide hole and illustrates a depth gauge;

FIG. 4A and FIG. 4B illustrates the relationship between the reamer and centering guide hole and the use thereof coupled to a driver;

FIGS. 5A and 5B illustrate the difference between prior art reamers (FIG. 5A) which lack a centering guide hole, and the instant invention (FIG. 5B) which takes advantage of the centering guide hole;

FIG. 6 is a perspective view of an implant positioned in accordance with the precision acetabular machining system of the present invention;

FIG. 7 is a cross-sectional view illustrating an implant according to the present invention, including central fixation means;

FIG. 8 is a perspective view of an implant positioned in accordance with the precision acetabular machining system of the present invention, and further including buttressing means posteriorly positioned upon the pelvis.

### DETAILED DESCRIPTION OF THE INVENTION

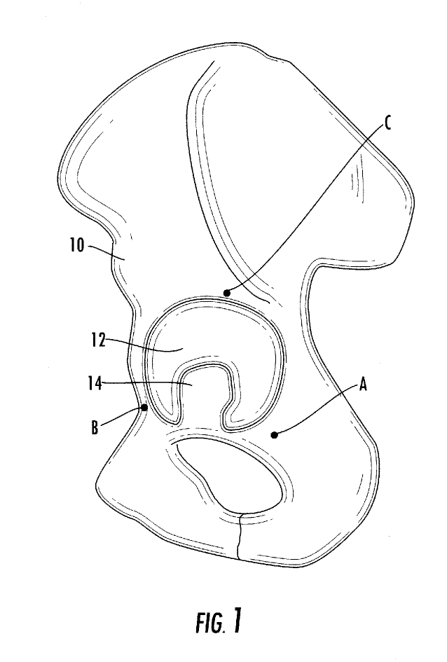

With reference to FIG. 1, the pelvis or ilium 10 is generally illustrated, specifically pointing out the lunate surface 12 and the foveal recess 14. The surgeon utilizes the foveal recess along with such landmarks as the lowest point of the acetabular sulcus of the ischium (Point A), the prominence of the superior pelvic ramus (Point B), and the most superior point of the acetabular rim (Point C) in determining the anatomical center.

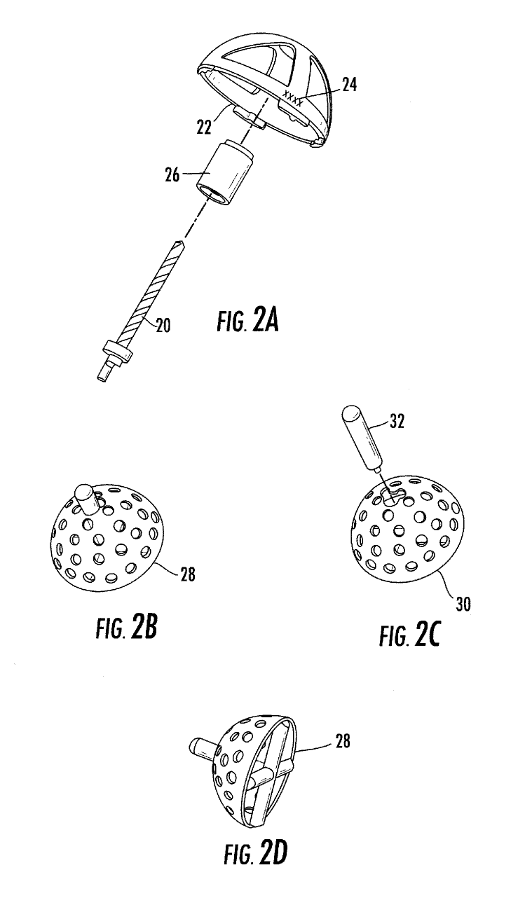

Referring to FIG. 2, A drill chuck 20 and drilling guide assembly 22 are shown; wherein the drilling guide 24 and drilling guide adaptor 26 are modular and when coupled precisely accommodate the acetabular guide drill chuck 20. Reamer assembly 28 is illustrated in varying perspectives showing the modular reamer basket 30 and guide peg 32.

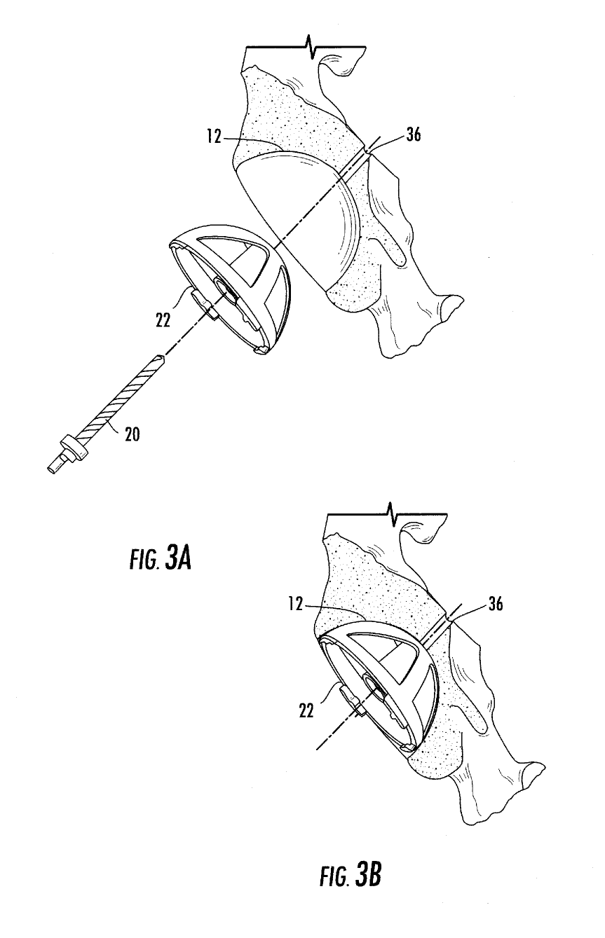

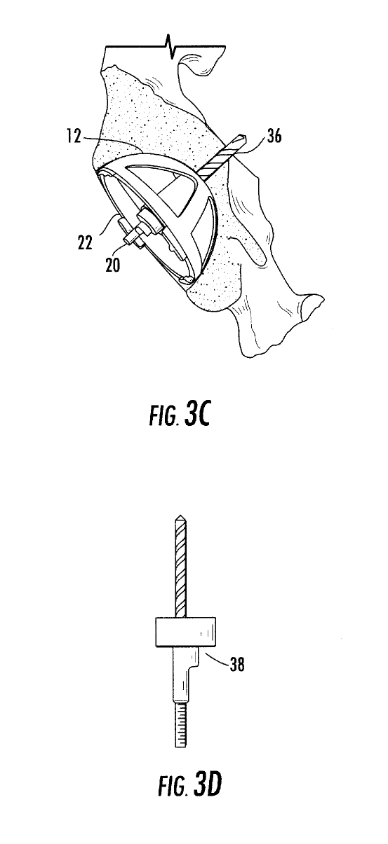

FIG. 3 further illustrates the acetabular socket 12, in cross-section, wherein the drilling guide assembly 22 and drill chuck 20, have been used in conjunction with a driver (not shown) to form the guide hole 36. Depth gauge 38 is used periodically to check depth so as to spare the greatest degree of bone possible.

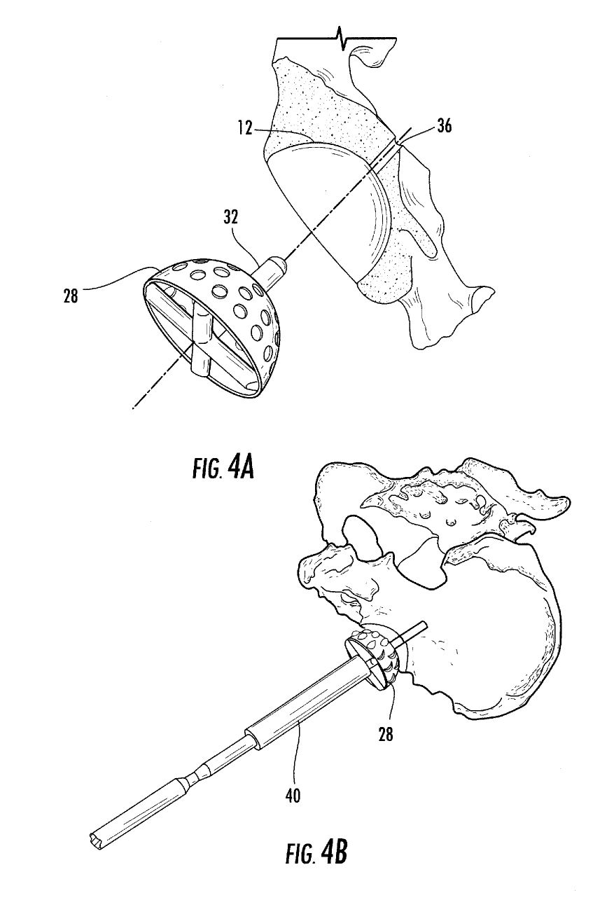

FIG. 4A further illustrates the acetabular socket 12, again in cross-section, and the relationship of the reamer assembly 28. FIG. 4B shows reaming of the acetabular socket 12 in accordance with the present invention, using the reamer assembly 28 coupled to driver 40.

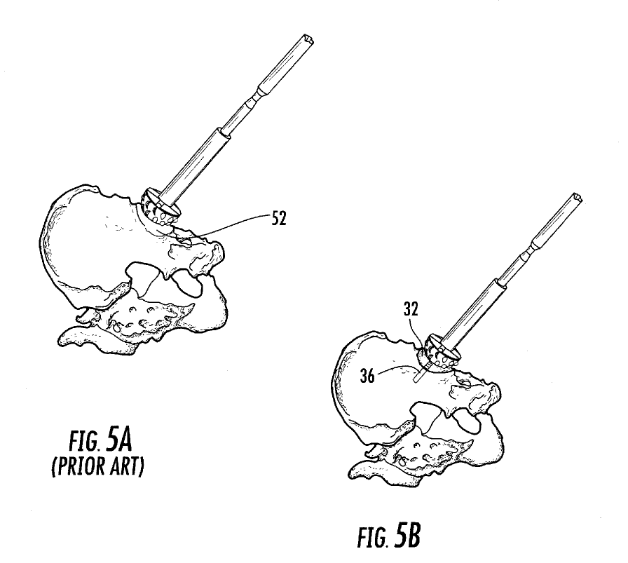

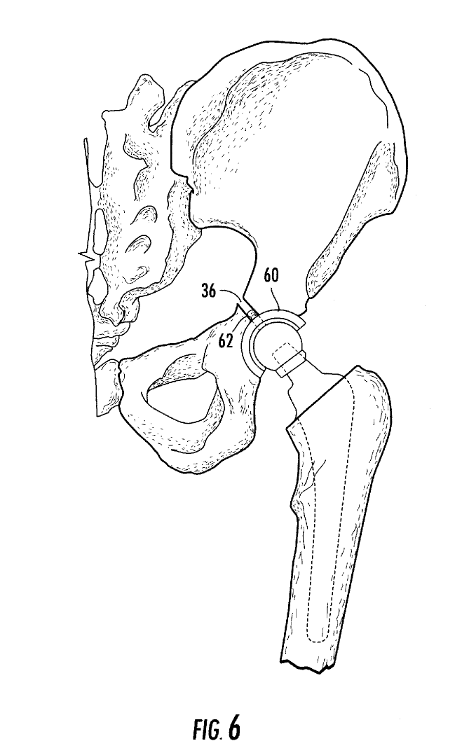

FIGS. 5A and 5B illustrate a prior art reaming process (FIG. 5A) wherein the lack of a guide hole and pin allow drift and oblong hole 52 formation, as compared to FIG. 5B wherein, in accordance with the PAMS of the present invention, the guide hole 36 and pin 32 combination provide for concentric reaming and formation of a precise hemispherical acetabular socket.

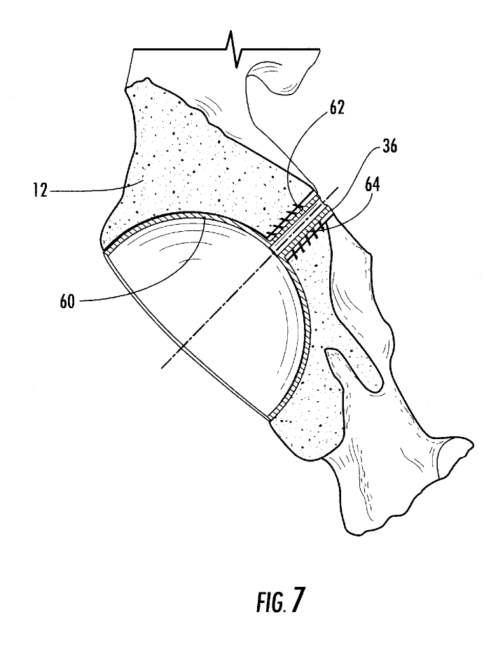

FIG. 6 illustrates a cross-sectional view which depicts implantation of a socket or implant 60 in accordance with the present invention. Because of the accuracy of the center pin guided reaming method, the implant is essentially hemispherical, and dose not have to be oversized or of varying radii in order to attain fixation. The essentially hemispherical profile permits fixation along the entire surface of the implant, as opposed to reliance upon edge loading. Thereby resulting in enhance bone ingrowth. It is noted that the relationship of the centering guide hole 36 and the pin 62 provided on the implant, permit rotation about the pin, during implantation, which provides adjustable version.

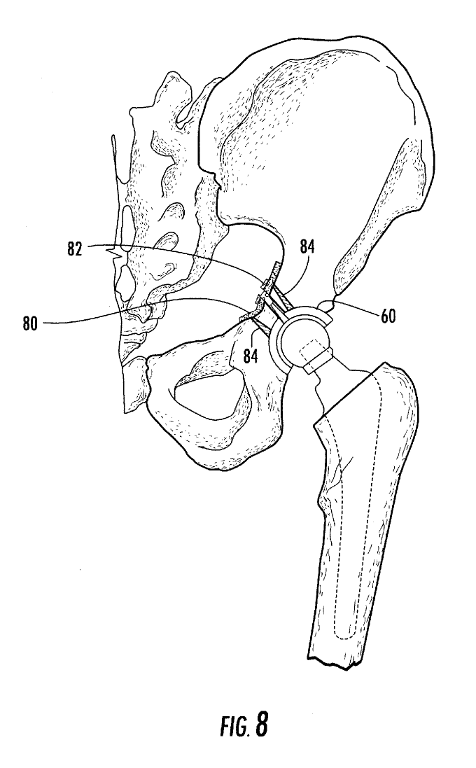

FIG. 7 is a further illustration of implant 60, wherein pin 62 has been modified to include hook-like appendages 64. This is merely illustrative, and it is understood that any retention means may be utilized for increasing fixation of the pin within the guide hole, such as a collapsible component, e.g. a collapsible or expandable anchoring device, a sleeve and screw combination, or the like.

FIG. 8 further illustrates an alternative application, wherein a buttressing member 80 is provided, which member may be made of a porous metal member which better enables bone ingrowth, such as a trabecular metal, for example a titanium foam or the like. This buttressing member 80 is positioned posterior of the acetabular socket, and a combination of fastening members, such as an arrangement wherein a backing plate or T-nut like device 82 is provided along the posterior side of the guide hole, and a screw or similar mating element is combined therewith to provide fixation. In a particular embodiment, this arrangement can enable optimal positioning and fixation within an otherwise fragile acetabulum, given that the socket 60 may be pulled into place, as opposed to having to be impacted in order to gain proper fixation. Additional screws 84 may be inserted at various points and provided with appropriate covers (not shown) to enable additional fixation and bone ingrowth where desired. It is contemplated that injection of demineralized bone, allograft, bone morphogenic protein or the like may be inserted within the guide hole or combined with the implant to aid ultimate fixation.

Materials contemplated for the bearing surfaces are any biocompatible material, such as titanium, porous metals, e.g. titanium foam, trabecular metal or the like, stainless steel, cobalt-chrome, ceramics, and the like. Where desired, a polyethylene liner or shell, inclusive of oxidant resistant polyethylene (E_POLY) or the like is further contemplated by the instant invention. Furthermore, any coating such as hydroxy apatite, plasma spray, porous coatings or combinations thereof, or the like, effective for enhancing bone ingrowth, are further contemplated for use by the instant invention.

### EXAMPLE

The procedure begins with a standard (anterior, posterior, transtrochanteric, etc) hip exposure of the acetabular socket. Next, the drill guide/reamers are used to properly size and center the socket followed by drilling the 5 mm bicortical centering hole. Then the depth gauge accurately measures the bone depth to the medial pelvic wall and the calibrated bullet guides with stops can be set to the desired reaming depth. Now, concentric reaming is begun to the correct size. Once this is done, the surgeon uses the acetabular implant driver/guide to accurately and safely seat the implant.

The resurfacing acetabular implant and instrumentation system has several novel features, including, albeit not limited to:
1. Self locking fixation with central axially aligned positioning pin and auxiliary fixation protrusion(s);
2. Novel advanced material technologies with advanced high endurance, articular surface and optionally including special bone inductive porous coatings for fixation, e.g. titanium foam, trabecular metal, or the like, allowing for ultra thin profile;
3. Ease of accurate and anatomically acceptable positioning e.g. about 40°±10° abduction and about 15°±10° of anteversion;
4. Provision of minimally invasive and maximally bone preserving, gauged and center guided instrumentation for accurate joint center placement;
5. Provision of maximal hip stability with wide range of head capabilities.

The resurfacing acetabular implant benefits from the utilization of low profile circular reamers centered on, for example, about a 3.5 mm central guide hole (low profile hemisphere) to remove any damaged cartilage and roughen the hard subchondral bone. The anatomic shape of the subchondral surface is identified by removing osteophyte from the foveal notch. Using the guide hole and depth gauge, only 2 mm of bone need be removed (unlike the 5-10 mm currently reamed through). By way of the guide hole this bone is removed concentrically without drift of the joint center.

When reaming is complete, the implant 60, as visualized in FIG. 6 for example, is loaded onto the impactor/positioner (not shown). Initial positioning of the guide is achieved via active or passive coupling to the center hole. As shown in the various Figures, the centrally located guide hole may simply act as a receiving means for a pin or peg protruding from the implant, thereby enabling the surgeon to insure positioning along the anatomical central axis. This procedure makes best use of the good cortical bone found at the junction of the medial wall and the ilium. Utilizing any form of manual or computer controlled form of guidance means, the surgeon then manipulates angulation of the implant central axis relative to the acetabulum, resulting in precise and central anatomic positioning of the acetabular resurfacing component. In order to eliminate any possibility of rotation of the implant about the central axis, at least one auxiliary locking means may be provided, e.g. screws or spikes, which engage the acetabular socket while permitting easy inset of the resurfacing implant. If additional stability is needed, then an alternative version, not illustrated, is contemplated, including, for example, one or two 6.5 mm screws, which can be drilled, tapped and placed. In a further contemplated embodiment, an anchoring element may be positioned within the central guide hole, which element is designed to mechanically engage the bone so as to provide a stable anchoring means for a fastener that is inserted through the central guide hole opening of the implant. Such a fastener, illustrated by, albeit not limited to a screw, collapsing anchor, or the like, provides both a means for fixation and a means for securely drawing the implant into the socket without requiring impact.

An alternative version utilizes an upstanding centering and depth defining post. The positioning post is graduated so as to act as a depth gauge to provide the surgeon with a visual landmark to assist in proper seating of the implant. The implant is lowered into the acetabular socket along the longitudinal axis of the post, and angulated appropriately as previously described. The graduations signal that the appropriate depth has been reached. At this point the post may be detached from the underlying anchoring means, which results in the provision of a threaded anchor fixedly engaged in the hard cortical bone structure, and suitable for attachment thereto of an anchoring screw, which serves the dual purpose of aiding fixation and sealing off the central hole.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of securing an acetabular cup to an anatomical center of an acetabulum comprising:
providing a drilling guide assembly including a drilling guide, drilling guide adapter, and drill chuck, effective for positioning and forming a guide hole in bone at said anatomical center of the acetabulum;
providing a hemispherical reamer assembly including a reamer basket and a centering guide peg, effective for reaming a concentric hemispherically-shaped hole within the acetabulum;
drilling a guide hole in the anatomical center of the acetabulum; reaming a hemispherical cavity into the acetabulum with said reamer assembly about said guide hole;
providing a hemispherical acetabular cup assembly including an acetabular cup and a centering peg adapted for insertion within said guide hole; and
fixing said hemispherical acetabular cup within said acetabulum;
whereby a firm overall fixation along the entire surface of the acetabular cup is achieved.

2. The method of claim 1, further comprising: inserting a liner into the acetabular cup.

3. The method of claim 2, wherein inserting a liner into the acetabular cup includes inserting a liner made of a polymeric material into the acetabular cup.

4. The method of claim 1, wherein said acetabular cup comprises a biocompatible material selected from the group consisting of titanium or an alloy thereof, cobalt chrome or an alloy thereof, stainless steel or an alloy thereof, ceramics and combinations thereof.

5. The method of claim 1, wherein an outside surface of said acetabular cup is porous.

6. The method of claim 5, wherein said porous outside surface is configured to enhance bone ingrowth.

7. The method of claim 1, wherein said acetabular cup includes a centering guide pin adapted for insertion within said guide hole.

8. The method of claim 7 wherein said centering guide pin is provided with fixation means for positive attachment to said guide hole.

9. A hemispherical acetabular cup for securement in an acetabulum comprising a hemispherical body and a central guide pin adapted for insertion within a guide hole formed in said acetabulum.

10. The hemispherical cup of claim 10, wherein said acetabular cup further includes fixation means for positive attachment to said acetabulum.

11. The hemispherical cup of claim 10, wherein said fixation means for positive attachment to said acetabulum are positioned within said guide hole.

12. The hemispherical cup of claim 9 wherein said cup comprises a biocompatible material selected from the group consisting of titanium or an alloy thereof, cobalt chrome or an alloy thereof, stainless steel or an alloy thereof, ceramics and combinations thereof.

13. The hemispherical cup of claim 9, wherein an outside surface of said cup is porous.

14. The hemispherical cup of claim 9, wherein said porous outside surface is configured to enhance bone ingrowth.

15. A reamer assembly adapted for precise forming of a hemispherical concentric hole within an acetabulum comprising in combination a reamer basket and a guide peg;
wherein said reamer assembly including said reamer basket and guide peg are adapted to provide precise hemispherical reaming of an acetabulum.

16. The method of claim 1 further including providing a depth gauge for determining a depth drilled into said bone.
